(19) United States
(12) Patent Application Publication
(10) Pub. No.: US 2025/0001111 A1
(43) Pub. Date: Jan. 2, 2025

(54) MULTI-CUFFED ENDOTRACHEAL TUBES

(71) Applicants: University of Pittsburgh - Of the Commonwealth System of Higher Education, Pittsburgh, PA (US); UPMC, Pittsburgh, PA (US)

(72) Inventors: Carl H. Snyderman, Pittsburgh, PA (US); Nikki Cwalina, Pittsburgh, PA (US); Brinden Elton, Dallas, TX (US); Maxwell Baxter, Cambridge, MA (US); Garrett N. Coyan, Pittsburgh, PA (US)

(73) Assignees: University of Pittsburgh - Of the Commonwealth System of Higher Education, Pittsburgh, PA (US); UPMC, Pittsburgh, PA (US)

(21) Appl. No.: 18/579,025

(22) PCT Filed: Jul. 14, 2022
(86) PCT No.: PCT/US2022/037085

§ 371 Date: Jan. 12, 2024

Related U.S. Application Data
(60) Provisional application No. 63/221,547, filed on Jul. 14, 2021.

(51) Int. Cl. A61M 16/04; A61M 16/00

(52) U.S. Cl. CPC A61M 16/0459; A61M 16/0003; A61M 16/044; A61M 16/0477; A61M 16/0486; A61M 2016/0027; A61M 2205/3327; A61M 2205/3331; A61M 2210/1032

(57) ABSTRACT

Disclosed herein are endotracheal tubes that have multiple inflatable cuffs to help form a seal for ventilation, to reduce unwanted migration of the tube relative to the patient, reduce focalized pressure from a single cuff, and to help prevent aspiration of materials between the outside of the tube and the tracheal walls. Some devices include first and second distal cuffs for placement within the trachea and a proximal third cuff for blocking the entrance to the trachea. The cuffs can include ridges or textures to enhance sealing. The device can also include inflation channels, vacuum channels, and pressure sensors.

### CROSS-REFERENCE TO RELATED APPLICATIONS

This application claims the benefit of U.S. Provisional Patent Application No. 63/221,547 filed Jul. 14, 2021, which is incorporated by reference herein.

### FIELD

This application relates to endotracheal tubes and similar tubes for placement within other hollow organs or cavities in the body.

### BACKGROUND

Conventional endotracheal tubes include a single inflatable cuff near the distal end of the tube to provide a seal for ventilation. Problems with such tubes include unwanted aspiration around the cuff resulting in ventilator-associated pneumonia, pressure necrosis from over-inflation of the single cuff, and unwanted tube migration (proximally and distally) with risk of hypoventilation or accidental extubation. Ventilator associated pneumonia is associated with significant morbidity and mortality and increases the cost of care very significantly per episode.

### SUMMARY

Disclosed herein are endotracheal tubes that have multiple inflatable cuffs to help form a seal for ventilation, to reduce unwanted migration of the tube relative to the patient, reduce focalized pressure from a single cuff, and to help prevent aspiration of materials between the outside of the tube and the tracheal walls. Some devices include first and second distal cuffs for placement within the trachea and a proximal third cuff for blocking the entrance to the trachea. The device can also include inflation channels, vacuum channels, and pressure sensors, among other features.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 shows an exemplary endotracheal tube with inflatable cuffs.

FIG. 2 shows a partial cross-section of a patient's airway with the endotracheal tube of FIG. 1 inserted into the trachea.

### DETAILED DESCRIPTION

Disclosed herein are endotracheal tubes (“devices”) that comprise a tubular portion with two or more external inflatable cuffs positioned around the tubular portion. The devices can also include inflation/deflation conduits running along the tubular portion from the cuffs and external inflation controllers coupled to the conduits for individually inflating and deflating the cuffs within the patient. The multiple cuffs can provide an enhanced seal for ventilation, distribute pressure more broadly, reduce unwanted proximal and distal migration of the tube and unplanned extubation resulting in patient morbidity/mortality, restrict materials from aspirating between the outside of the tube and the tracheal walls and subsequent ventilator-associated tracheobronchitis/pneumonia, avoid complications from continuous subglottic suction, and/or can provide other advantages over conventional single-cuff tubes.

Any of the devices, methods, and other features disclosed herein can be combined in any combination, and can be combined with any of the devices methods, and other features (such as one or more baffles) disclosed in U.S. Provisional Patent Application No. 63/221,572 filed Jul. 14, 2021, entitled “ENDOTRACHEAL TUBES WITH BAFFLES,” and the co-pending International PCT Application filed on the same day as this application, also entitled “ENDOTRACHEAL TUBES WITH BAFFLES,” which are incorporated by reference herein in their entirety.

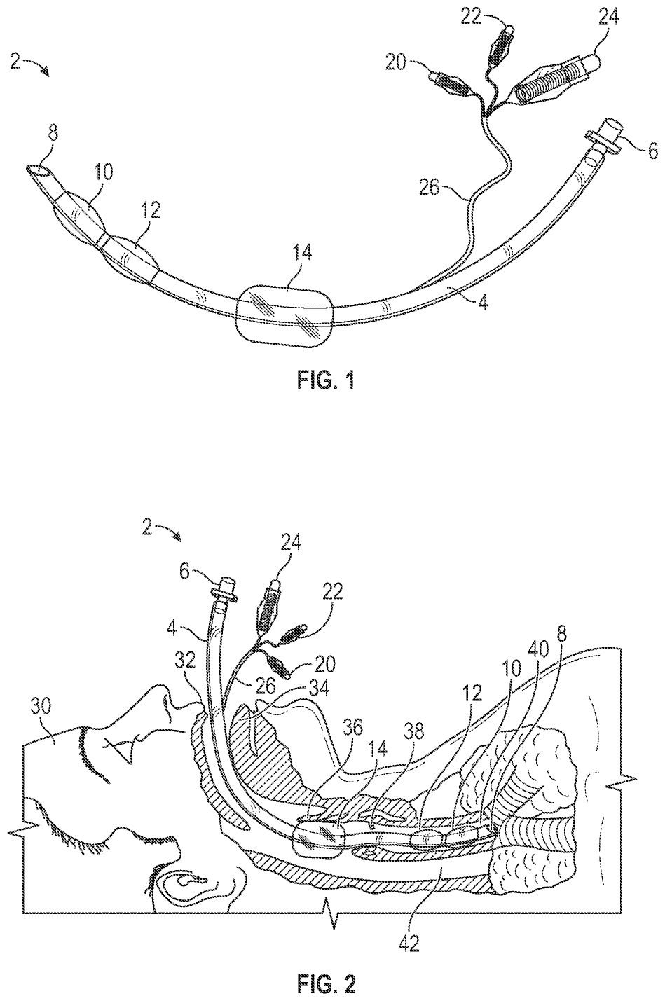

FIG. 1 shows an exemplary endotracheal tube (also referred to as the “device”) 2 that comprises a tubular portion 4, a proximal adaptor 6, a distal opening 8, a relatively large proximal inflatable cuff 14, two smaller distal inflatable cuffs 10, 12, and three inflation controllers 20, 22, 24 fluidly coupled to the cuffs via conduits 26 running along the tubular portion adjacent to the main ventilation channel. FIG. 2 shows a partial cross-section of a patient 30 illustrating the device 2 inserted into the trachea 40. As shown in FIG. 2, the device 2 is inserted through the mouth 32, over the tongue 34, past the epiglottis 36, through the larynx 38, and into the trachea 40. The esophagus 42 is also shown for reference.

The smaller, distal cuffs 10, 12 inflate to conform to the shape of the trachea 40 at a location between the larynx and the bronchi. The cuffs 10, 12 engage the walls of the trachea 40 to hold the device 2 in the desired position, such as with the distal outlet 8 proximal to the bronchi. The cuffs 10, 12 create a seal to support ventilation while blocking aspiration between the tubular portion 4 and the trachea 40 and resisting unwanted migration of the device. The two separate cuffs can distribute inflation pressure more broadly along a longer section of the trachea compared to a single cuff doing the same work. Some embodiments can include only one distal cuff, and some embodiments can include three or more distal cuffs. The distal cuffs can be the same size or different sizes, both in axial length and diameter.

The larger, proximal cuff 14 is positionable above the trachea adjacent to the epiglottis and helps prevent distal migration of the device, helps block the entrance to the trachea between the epiglottis 36 and the larynx 38 to reduce aspiration, and redirects materials in the airways toward the esophagus. The proximal cuff can be larger in diameter than the distal cuffs. In some embodiments, the third cuff can have different shapes that conform to the anatomy of the hypopharynx and supraglottic larynx to secure the tube and divert secretions.

The controller 20 inflates and deflates the cuff 10, the controller 22 inflates and deflates the cuff 12, and the controller 24 inflates and deflates the larger, proximal cuff 14. In some embodiments, a single inflation controller can be associated with two or more of the cuffs, either being commonly fluidly coupled to the two or more cuffs such that they are inflated/deflated together, or with the controller having two or more different inflation mechanisms. For example, in some embodiments, a single inflation controller can be used to inflate the two distal cuffs at the same time, and in some embodiments a single inflation controller can be coupled to all three cuffs and can include three different inflation mechanisms within the single controller.

The inflation controllers can be manually operated, machine operated, or otherwise operated. For example, in the device 2, each of the controllers 20, 22, 24 includes its own manual spring-actuated hand pump, such that an operator can depress a pump button repeatedly to add incremental amounts of air or other fluid (gas or liquid) into the cuffs. In other embodiments, a pneumatic or hydraulic pump can be used to inflate the cuffs. In other embodiments, other types of pumps can be used. The cuffs are inflated with ambient air, other gases such as oxygen, or liquid.

In some embodiments, the device can also include pressure sensors on, inside of, or adjacent to the cuffs to monitor cuff pressure and/or detect improper placement and/or migration in real-time. Such sensors can be coupled via wires or wirelessly to an external device, such as a monitor that provides feedback to the user.

In some embodiments, the device can include one or more vacuum channels extending along the tubular portion and coupled to openings in the wall of the tubular portion adjacent to the cuffs. Such openings can be located distal to the distal-most cuff, between the distal cuffs, or between the proximal cuff and the distal cuffs. The one or more vacuum channels can be coupled at their proximal ends to an external vacuum source to cause suction at the openings adjacent to the cuffs, in order to suck out any built-up fluids or other materials around the cuffs. This can further help prevent aspiration and infection. The one or more vacuum channels can run alongside the primary ventilation channel and the cuff inflation channels in a parallel or coaxial manner.

In some embodiments, the tubular portion can be reinforced with wires, bars, stents, ribs, rings, or other relatively rigid materials to prevent kinking, compression, or other unwanted deformation of the tubular portion.

Some embodiments can include an umbrella-shaped shield or cover for the supraglottic airway in the proximal cuff position (either in place of the larger proximal cuff, or in addition to it and proximal to it). Such a shield can help divert oropharyngeal secretions and other materials away from the trachea and reduce aspiration.

In some embodiments, the tubular portion can comprise a double lumen that forks into two distal end portions, or forks, for independent ventilation of each lung. Each fork can terminate in or adjacent to a respective one of the bronchi. In such embodiments, each fork can have its own one or two or more distal inflatable cuffs, similar to the cuffs 10, 12. In some embodiments, a larger, proximal cuff is located around the tube before the fork, and one or more smaller distal cuffs are located around each fork.

In some embodiments, the tubular portion and the cuffs can be co-formed of a common material. In other embodiments, the tubular portion and the cuffs can be separately formed and later coupled together. For example, the cuffs and the tubular portion can be formed of different materials that have different properties. In some embodiments, the tubular portion and/or the cuffs can comprise composite construction, being constructed of a combination of two or more different materials. Such materials can comprise various polymeric materials, metallic/alloy materials, reinforcing components, outer coatings, radiographic elements, visual markings, etc.

In some embodiments, the tubular portion can be made of a single material with variable thickness along its length and therefore variable rigidity and flexibility along its length.

In some embodiments, the cuffs and/or outer surface of the tubular portion can be coated or impregnated with various substances, such as an antimicrobial agent, non-stick material, anticoagulation material, etc.

In some embodiments, the multiple cuffs create closed compartments between the cuffs that limit formation of biofilm and other materials on the tube portion. In some embodiments, the multiple cuffs create closed compartments that prevent air escape and limit oxygen desaturation.

An exemplary method of use comprises inserting any of the herein described endotracheal tubes into the trachea, then inflating the most distal cuff as desired, then inflating the second most distal cuff as desired, and then inflating the third most distal cuff as desired, etc., until all the cuffs are inflated as desired. Once the cuffs are inflated, the tube is secured within the patient and ventilation can be performed. Suction can be applied periodically while the cuffs are inflated to remove materials around the cuffs. Cuff inflation pressure and cuff position can also be monitored via sensors at the cuffs. When it is desired to remove the tube from the patient, the cuffs can be deflated in any manner and any order, such as in reverse order with the most proximal cuff first, the second most proximal cuff second, etc., and the distal most cuff last, before the device is retracted proximally out of the patient.

In addition to the features disclosed elsewhere in this disclosure, any embodiment can comprise any one or more of the following features, in any combination with other features. In some embodiments, a proximal umbrella covers the supraglottic larynx and protects the vocal cords and subglottic space from aspiration of blood and secretions. In some embodiments, a proximal umbrella clears blood and other secretions from the hypopharynx/upper airway with removal. In some embodiments, a proximal umbrella diverts tubes and secretions toward the esophagus, promoting passage into the esophagus and preventing unintended passage into the larynx and trachea with potential trauma. In some embodiments, horizontal ridges of thickened cuff material of varying circumference interrupt and prevent the formation of longitudinal creases that allow aspiration.

### General Considerations

Characteristics, materials, and other features described in conjunction with a particular aspect, embodiment, or example of the disclosed technology are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.” As used herein, the term “coupled” generally means physically coupled or linked and does not exclude the presence of intermediate elements between the coupled items absent specific contrary language.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the full scope of the following claims. We therefore claim all that comes within the scope of these claims and their equivalents.

1. An endotracheal tube comprising:
a tubular portion;
a proximal adaptor at a proximal end of the tubular portion;
a distal opening at or adjacent to a distal end of the tubular portion;
a first inflatable cuff positioned around the tubular portion and a second inflatable cuff positioned around the tubular portion proximal to the first cuff, wherein the first and second cuffs are configured to contact an inner wall of a patient's trachea when the endotracheal tube is inserted into the trachea and the first and second cuffs are inflated, such that the first and second cuffs resist migration of the endotracheal tube relative to the trachea and block aspiration between the tubular portion and an inner wall of the trachea; and
a third inflatable cuff positioned around the tubular portion proximal to the second cuff, wherein the third cuff is larger in diameter than the first and second cuffs when inflated, and wherein the third cuff is configured to be positioned adjacent to the patient's epiglottis to block the entrance to the trachea.

2. The endotracheal tube of claim 1, wherein the tubular portion comprises a primary channel fluidly coupled to the distal opening, a first inflation channel fluidly coupled to the first cuff, a second inflation channel fluidly coupled to the second cuff, and a third inflation channel fluidly coupled to the third cuff; wherein the first, second, and third inflation channels extend parallel to the primary channel proximally from their respective cuffs.

3. The endotracheal tube of claim 2, further comprising a first inflation controller coupled to a proximal end of the first inflation channel, a second inflation controller coupled to a proximal end of the second inflation channel, and a third inflation controller coupled to a proximal end of the third inflation controller.

4. The endotracheal tube of claim 1, wherein the first, second, and third cuffs are independently inflatable and deflatable.

5. The endotracheal tube of claim 1, wherein the first and second cuffs are axially spaced apart such that an open compartment is formed between the first and second cuffs and between the tubular portion and the trachea when the cuffs are inflated within the trachea.

6. The endotracheal tube of claim 1, further comprising pressure sensors on, in, or adjacent to the first, second, and third cuffs to monitor cuff pressure and detect improper placement and/or migration in real-time.

7. The endotracheal tube of claim 1, wherein the tubular portion comprises one or more vacuum channels extending along the tubular portion and coupled to openings in a wall of the tubular portion adjacent to the first and second cuffs for drawing materials out of the trachea from around the cuffs.

8. The endotracheal tube of claim 1, wherein the first and second inflatable cuffs have different shapes and contours.

9. A method comprising inserting the endotracheal tube of claim 1 into the patient's trachea, then inflating the first cuff, then inflating the second cuff, and then inflating the third cuff.

10. The method of claim 9, wherein the first and second cuffs are fully inflated before the third cuff begins to be inflated.

11. The method of claim 9, wherein all of the first, second, and third cuffs are partially inflated before any of them are fully inflated.
